(19) United States
(12) Patent Application Publication
(10) Pub. No.: US 2006/0014300 A1
(43) Pub. Date: Jan. 19, 2006

(54) Sensor for detection and identification of biological particles

(76) Inventors: Scott M. Maurer, Haymarket, VA (US); Ryan C. Brewer, Bristow, VA (US); Larry D. Jackson, Manassas, VA (US); Kevin J. Kofler, Bristow, VA (US)

Correspondence Address: DEMONT & BREYER, LLC, SUITE 250, 100 COMMONS WAY, HOLMDEL, NJ, 07733, US

(21) Appl. No.: 10/891,805

(22) Filed: Jul. 15, 2004

(51) Int. Cl. G01N 33/543; C12M 1/34

(52) U.S. Cl. 436/518; 435/287.2

(57) ABSTRACT

The illustrative embodiment of the present invention is a system and a method for the detection and limited identification of biological agents. The system is small, light weight, requires little power to operate and uses few consumables. The system can be configured for use in either stationary or mobile applications. The system incorporates elements that enable it to obtain an air sample, extract +particulates from the air sample onto a stationary-phase collection media, exposes the particulates to electromagnetic radiation, and monitor for fluorescent emissions. To the extent that fluorescent emissions are detected and exceed a predetermined value, an alarm is triggered. In some embodiments, in addition to performing real-time analyses on the extracted particulates, the collection media is removed from the system and the sample is subjected to more detailed analysis via additional equipment (e.g., pcr, etc.). Various sample-collecting regions on the collection media are “time stamped” or “location stamped” so that it can determined when and/or where each sample that is being analyzed “off-line” was obtained.

### STATEMENT OF RELATED CASES

This case is related to co-pending U.S. patent applications Ser. Nos. ______ (Attorney Docket Nos. 711-016, 711-018, 711-019, and 711-020), which were filed on even date herewith and are incorporated herein by reference.

### BACKGROUND OF THE INVENTION

Biological warfare is the intentional use of microorganisms and toxins of microbial, plant or animal origin to produce diseases and/or death in humans, livestock and crops. To terrorists, biological warfare is attractive because bio-weapons have relatively low production cost, it is relatively easy to obtain a wide variety of disease-producing biological agents, bio-weapons are non-detectable by routine security systems, and bio-weapons are easily transportable.

Unlike relatively mature radiation- and chemical-detection technologies, early-warning technology for biological agents is in its infancy. Most known bio-detection systems are “flow-through,” wherein individual particles that are contained in a flowing stream (e.g., air, etc.) are interrogated in an optical cell. Interrogation is typically performed using high-power lasers. The flowing stream, and hence the particles, have an extremely low residence time in the optical cell. As a consequence, the laser samples only a portion of the stream, must be relatively high power to provide an appropriate signal-to-noise ratio, and must be operating constantly to ensure detection.

Furthermore, some bio-detection systems use consumables, such as buffered saline solutions, antibodies, assay strips, reagent solutions, cleansing solution and antibodies. Most of these consumables have a specific shelf life, which creates a logistical burden. Furthermore, these consumables are typically unable to withstand demanding thermal requirements in theater. Also, many current bio-detection systems are large, heavy, and consume large amounts of power.

The drawbacks of prior-art bio-detection systems, as described above, significantly limit their usefulness in the field.

### SUMMARY

The illustrative embodiment of the present invention is a sensing system and method for the detection and limited identification of biological agents. Unlike many prior-art bio-detection systems, the sensing system is small, light weight, requires little power to operate and uses few consumables. The system can be configured for use in either stationary or mobile applications.

The principle of operation for the sensing system is that many biological agents “fluoresce” when excited by radiation that has an appropriate wavelength, which is typically within or near the ultraviolet range. “Fluorescence” is the radiation that is emitted from a biological agent (or other substances) when it is excited as described above. What occurs at a molecular level is that the substance absorbs a photon of electromagnetic radiation, which causes an electron in the substance to move from a low energy state to a higher one. When the electron returns to a lower energy state, a photon is emitted. This photon is fluorescent radiation.

Since many types of biological agents fluoresce under ultraviolet light, the detection of fluorescent emissions from a sample that has been exposed to radiation having a wavelength in or near the ultraviolet range indicates that biological agents might be present. This is the detection function of the sensing system; some embodiments of the sensing system also provide a limited identification function as well.

Regarding identification, different biological agents contain different fluorescing organic substances (e.g., differing in amount or type). As a consequence, the peak intensity of the fluorescence emissions and/or characteristic fluorescent spectra for these different biological agents will be different. This attribute, among any others, provides a basis for at least limited identification of biological agents.

Briefly, in a method in accordance with the illustrative embodiment:
an air sample is obtained;
particulates are extracted from the air sample;
the particulates are exposed to electromagnetic radiation (typically in the ultraviolet to blue range of wavelengths); and
the particulates are monitored for fluorescent emissions.

To the extent that fluorescent emissions are detected and exceed a predetermined value, it is indicative that a biological attack might be in progress or might have occurred. Characteristics of the fluorescent emissions (e.g., wavelength, intensity, etc.) can be used to identify a biological agent that has been detected by the system.

A sensing system in accordance with the illustrative embodiment comprises an interrogation cell, which has:
A stationary-phase collection media for extracting and retaining particulates, including biological agents, from an air sample. The collection media includes a plurality of sample-collecting regions.
A device or arrangement that is capable of moving the collection media or redirecting the flow of air so that sample-collecting regions are selectively and individually exposed to a flow of air.
A source of electromagnetic radiation for exposing particulates that have been retained in the collection media. If the retained particulates include biological agents, they will fluoresce when exposed to electromagnetic radiation having an appropriate wavelength. Wavelengths within a range of about 250 to about 500 nanometers are appropriate for causing fluorescence in many biological agents. In the illustrative embodiment, the source of electromagnetic radiation is one or more light-emitting diodes (“LEDs”).
A detector, such as a photodetector, for monitoring fluorescent emissions. The detector must be sensitive to the wavelengths of radiation at which biological agents fluoresce. The peak wavelength(s) of fluorescent emissions from biological agents of interest is typically in the range of about 300 to about 600 nanometers.

In addition to the interrogation cell, the sensing system also includes control/data-acquisition/data-processing circuitry. This circuitry is capable of implementing the following functions, among others:
Controlling the operation of the source of electromagnetic radiation, including an ability to intermittently activate the source.
Controlling the operation of the detector including activating the detector and acquiring data from the detector.
Controlling the operation of the device that is capable of moving the collection media or redirecting the flow of air.
Signal processing. A signal generated by the photodetector is processed to:
   detect: determine if a biological agent is present in the air sample;
   quantify: estimate the amount of biological agent present, if any;
   assess: determine if the amount of a biological agent present is indicative of a biological attack or otherwise poses a risk to the health of the local population, livestock, etc.; and
   identify: provide a limited identification of a biological agent that is detected.

In some embodiments, in addition to performing real-time analyses on the extracted particulates, the collection media is removed from sensing system 100 and is subjected to more detailed analysis (e.g., pcr, etc.). The various sample-collecting regions on the collection media are “time stamped” or “location stamped” so that it can determined when and/or where each sample that is being analyzed was obtained. In such embodiments, sensing system 100 includes a device for associating each sample-collecting region that has been exposed to an air sample with at least one of either a time or a location.

### BRIEF DESCRIPTION OF THE DRAWINGS

FIG. 1 depicts a sensing system for the detection of biological agents in accordance with the illustrative embodiment of the present invention.

FIG. 2 depicts a method for the detection of biological agents in accordance with the illustrative embodiment of the present invention.

FIG. 3 depicts an interrogation cell of the sensing system of FIG. 1.

FIG. 4 depicts a top view of an illustrative collection media, wherein said media is divided, at least conceptually, into four sample-collecting regions.

FIG. 5 depicts a first arrangement for exposing, at a different time, each sample-collecting region of the collection media depicted in FIG. 4.

FIG. 6 depicts a second arrangement for exposing, at a different time, each sample-collecting region of the collection media depicted in FIG. 4.

FIG. 7 depicts a top view of a shutter that is used in the second arrangement, which is shown in FIG. 6.

FIG. 8 depicts a third arrangement for exposing, at a different time, each sample-collecting region of the collection media.

### DETAILED DESCRIPTION

The illustrative embodiment of the present invention is a sensing system and method for the detection and limited identification of biological agents. In some embodiments, the sensing system is very light and quite small, fitting in an enclosure that is about 1 inch×1 inch×2 inches. The system can be configured for use in either stationary or mobile applications.

Biological agents of interest here typically have a size that is in a range of hundreds of nanometers (e.g., for viruses, etc.) to a few microns (e.g., for bacteria, etc). Typical biological agents of interest include, for example, anthrax (1×2 micron), plague (0.5×1 micron), tularemia (0.5×1 micron), and small pox (200×250×250 nanometers). The illustrative embodiment of the present sensing system is capable of detecting particles in this size range. In some variations of the illustrative embodiment, the sensing system is configured to detect smaller biological agents, and in yet some additional variations, the sensing system is configured to detect larger biological agents.

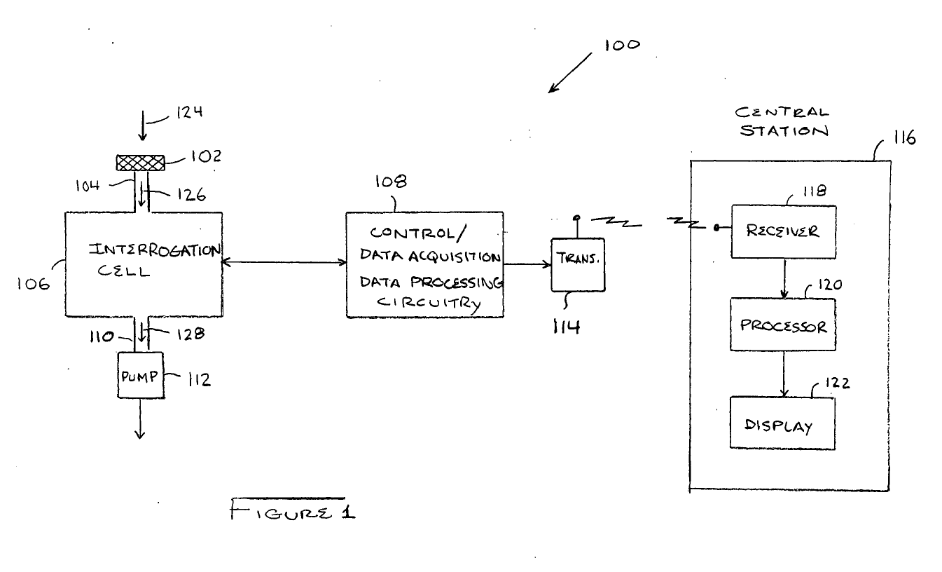

FIG. 1 depicts sensing system 100 in accordance with the illustrative embodiment of the present invention. Sensing system 100 comprises interrogation cell 106, control/data acquisition/data processing circuitry 108, and central station 116, interrelated as shown.

A sample of air is obtained from the ambient environment for interrogation within interrogation cell 106. If sensing system 100 is stationary, then air is drawn through the sensing system by pump 112 or other similar device (e.g., a device that generates a suction flow, etc.). If the sensing system is moving (e.g., disposed on a vehicle, attached to a device that rotates the system, etc.), then pump 112 might not be necessary as a function of the speed at which sensing system 100 is moved.

In the illustrative embodiment, the sample of air, identified as flow 124 in FIG. 1, is filtered before it enters interrogation cell 106. In the illustrative embodiment, filtration is performed by filter 102, which is disposed upstream of cell inlet line 104.

Filter 102 prevents large particulate matter from entering interrogation cell 106. If large particulates were to enter interrogation cell 106, they might clog the interrogation cell, thereby shortening run time. In some embodiments, filter 102 filters particulate matter that is larger than about 50 microns. At this size, filter 102 will trap large dust particles, insects, and the like. Since, as described above, most biological agents of interest are much smaller than 50 microns, they will readily pass filter 102 and enter interrogation cell 106.

Filter elements suitable for use in the illustrative embodiment as filter 102 have a 50-micron pore structure and include, without limitation:

| | | |
|---|---|---|
| glass micro-fiber | paper | anodized aluminum |
| Teflon ™ -based materials | stainless steel | polymers/plastics. |

At least some of these filter elements are available from Donaldson Company of Minneapolis, Minn.; the other elements are available from any of a variety of commercial suppliers.

As an alternative to filter 102, a micro virtual impactor concentrator (micro-VIC®) can be used. The micro-VIC®, which is available from MesoSystems Technology, Inc. of Albuquerque, N. Mex., utilizes inertial effects to discharge and separate larger particulates from relatively smaller biological agents. Another alternative to a filter is a rotating-arm impactor.

Filtered flow 126 of air is conducted via cell inlet line 104 to interrogation cell 106. As described more fully later in this specification, particulates are removed from filtered flow 126 and interrogated in the interrogation cell. After passing through interrogation cell 106, substantially particulate-free flow 128 of air is expelled from sensing system 100 via cell outlet line 110.

The operation of interrogation cell 106 is controlled by control/data acquisition/data processing circuitry 108. Information that is obtained from the interrogation of the particulates is transmitted to station 116, which, in the illustrative embodiment, is remote from interrogation cell 106. In the illustrative embodiment, transmission is performed wirelessly via transmitter 114. The transmitted information is received by receiver 118, is processed as required in processor 120, and is displayed on display 122. In some alternative embodiments, control/data acquisition/data processing circuitry 108 is wired to station 116.

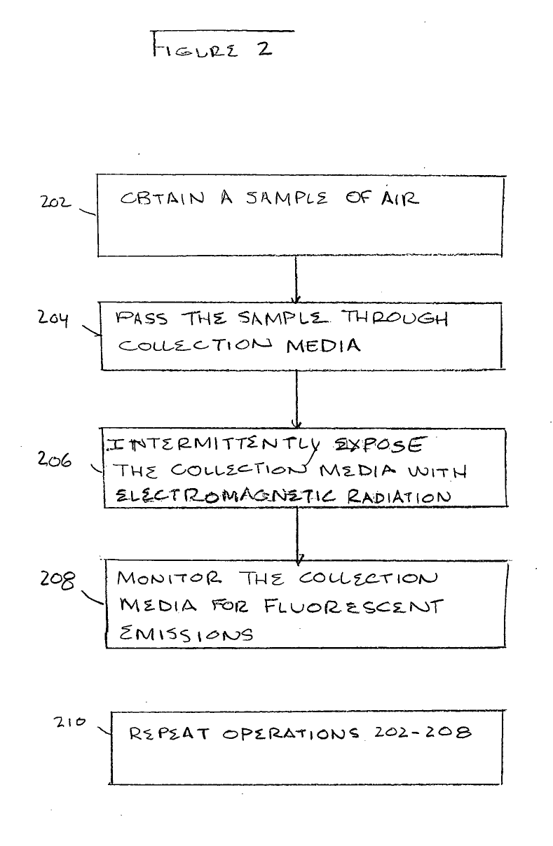

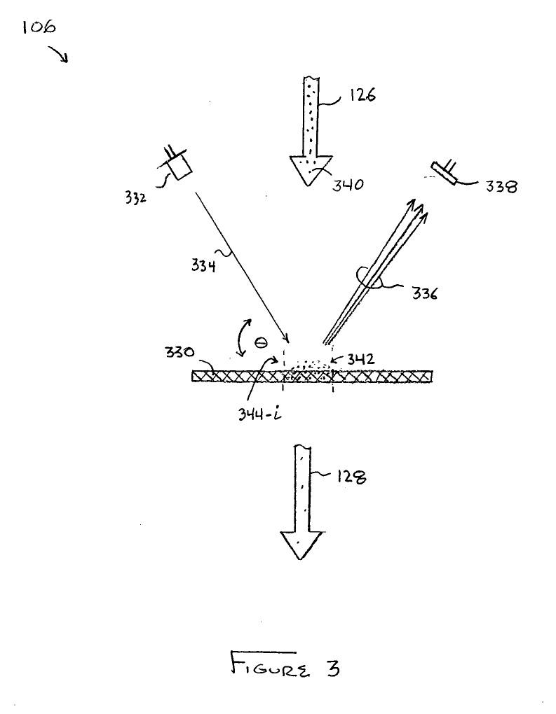

Having provided an overview of sensing system 100, description of the operation and structure of interrogation cell 106 is now provided. The description proceeds with reference to FIG. 2, which depicts method 200 for detection of biological agents, and FIG. 3, which depicts the structure of interrogation cell 106.

The operations of method 200 include:
obtaining a sample of air (operation 202);
+passing the sample of air through collection media, wherein the collection media is capable of retaining particulates that are contained in the sample of air (operation 204);
exposing the collection media to electromagnetic radiation (operation 206);
monitoring the collection media for fluorescent emissions (operation 208); and
repeating operations 202-208.

Operation 202 of method 200 recites “obtaining a sample of air.” A purpose of operation 202 is to provide a sample of air for interrogation by interrogation cell 106.

Operation 204 of method 200 recites “passing the sample through collection media, wherein the collection media is capable of retaining particles contained in the sample.” A purpose of operation 204 is to extract any biological agents that might be contained within the air sample (i.e., filtered air sample 126) so that they can be interrogated.

Referring now to FIG. 3, filtered air flow 126 is directed to one of a plurality of sample-collecting regions 344-i of stationary-phase collection media 330. (Only one such sample-collecting region 344-i is depicted in FIG. 3; see FIGS. 4-8 and the accompanying description.) The collection media comprises a stationary phase that is physically adapted to trap at least about 99 percent of particulates 340 that remain in filtered air flow 126 and have a size in the range of interest for biological agents (i.e., about 0.3-5 microns). Particulates that are retained by collection media 330 compose sample 342. Interrogation cell 106 can be provided with stationary-phase collection media 330 having a more definitive rating to the extent that it is intended to monitor a specific type of threat (i.e., a particular biological agent).

Stationary-phase collection media 330 suitable for use in conjunction with sensing system 100, as a function of the biological agents of interest, includes:
HEPA/ULPA glass microfiber filtration media that is rated at >99.7% removal efficiency for particulates at 0.3 microns.
PTFE/PFA/PFE (i.e., Teflon®-based) filtration media that is rated at >99% for particulates at 0.3 microns.
Paper filtration media that is rated at >99% for particulates at 0.3 microns.
Stainless Steel filtration media that is rated at >99% for particulates at 1 micron.
Anodized Aluminum filtration media that is rated at >99% for particulates at 1 micron.
Other types of filtration media such as plastics and other polymers that are rated at >99% for particulates at 0.3 microns.

As previously indicated, after passing through collection media 330, the now substantially particulate-free flow 128 of air is expelled to the ambient environment via cell outlet line 110.

In some embodiments, even those in which the sensing system 100 is mobile, an appropriately-valved pump is included in the system and used to reverse the flow of air through collection media 330. Reversing the flow of air removes at least some of the material (i.e., particulates 340) that has been retained by collection media 330. Reversing the flow in this manner might be necessary if the collection media becomes clogged. Alternatively, this technique can be used to establish a new interrogation baseline (e.g., for fluorescent emissions, etc.).

Operation 206 of method 200 recites “exposing the collection media to electromagnetic radiation.” A purpose of this operation is to excite to fluorescence any biological agents that have been trapped by collection media 330.

With continuing reference to FIG. 3, interrogation cell 106 includes a source of electromagnetic radiation, which in the illustrative embodiment is LED 332. Electromagnetic radiation 334 generated by LED 332 is directed toward sample 342 on collection media 330. Since most biological agents of interest are excited by wavelengths between about 250 to 500 nanometers (i.e., the ultraviolet to blue range of wavelengths), the peak emission wavelength of LED 332 should be within this range. LEDs emit radiation over a range of wavelengths. Typically, one wavelength will contain more energy than any other single wavelength. That one wavelength is the “peak emission wavelength.”

In some embodiments, LED 332 does not remain on continuously; rather, it is pulsed on and off. LED 332 is controlled for intermittent operation via control/data acquisition/data processing circuitry 108. In comparison with an always-on, laser-based system, the use of an LED, especially in a pulsed mode, consumes far less power. For example, when implemented without pump 112, the average power consumption of sensing system 100 is expected to be about 100 mW at 5V. The sensing system is adaptable for battery operation, as desired, at 6, 12 or 24 volts DC.

LED 332 can be positioned at any out-of-plane angle θ relative to collection media 330. The angle θ is typically in the range of 0 to 90 degrees. More typically, angle θ lies between 45 to 60 degrees.

Operation 208 of method 200 recites “monitoring the collection media for fluorescent emissions.” A purpose of this operation is to detect the presence of biological agents.

Referring again to FIG. 3, system 100 includes at least one photodetector 338 for monitoring fluorescent emissions 336 from any biological agents present in sample 342 on collection media 330. In the illustrative embodiment, the photodetector is a photodiode. Photodetector 338 must be sensitive to the wavelengths at which biological agents fluoresce. Most biological agents of interest fluoresce at wavelengths that are within the range of about 300 to about 600 nanometers. For example, tryptophan (an amino acid that is typically found in animal proteins or bacteria) has a peak emission at about 330 nanometers, NADH (usually associated with growth media and yeast grown products that are used for culturing organisms) has a peak at around 450 nanometers and flavins (again associated with growth media) have a peak at around 560 nanometers. As a consequence, photodetector 338 should be sensitive to wavelengths in this range.

Interrogation cell 106 can be arranged to have any one of a variety of configurations, including:
Single LED and single photodetector;
Single LED and photodetector array or multiple individual photodetectors;
Multiple LEDs and single photodetector;
Multiple LEDs and photodetector array or multiple individual photodetectors.
   These configurations of interrogation cell 106 are described in detail in applicants' co-pending U.S. patent application Ser. No. ______ (Atty. Dkt. 711-016).

Control/data acquisition/data processing circuitry 108 (FIG. 1) controls much of the operation of interrogation cell 106. In this context, this circuitry, which in some embodiments includes a processor and memory, is capable of:
driving LED(s) 332; and
capable of intermittently pulsing LED(s) 332; and
enabling photodetector(s) 338.
   As described later in this specification, circuitry 108 is also capable, in conjunction with a drive system (e.g., motor, etc.), of moving the collection media or redirecting the flow of air.

Photodetector 338 generates a signal(s) in known fashion when it receives fluorescent emissions 336. The signal(s) contains information pertaining to the fluorescent emissions. For example, in some embodiments, the signal(s) is indicative of the wavelength(s) of the fluorescent emissions and the intensity of those emissions. This information can be used to develop a relative “particulate” (i.e., biological agent) count as a function of wavelength.

Control/data-acquisition/data-processing circuitry 108 receives the signal(s) from the photodetector (representative of the fluorescent emissions) and performs one or more of the following tasks:
stores a representation of the signal; and/or
partially processes the signal; and/or
fully processes the signal; and/or
transmits (in conjunction with transmitter 114), to central station 116:
   a representation of the signal; or
   a representation of the signal as well as data obtained from partially processing the signal; or
   a representation of the signal as well as data obtained from fully processing the signal; or
   only the information obtained from processing the signal.
      In some embodiments, operation 208 (i.e., monitoring the collection media for fluorescent emissions) also includes the task(s) described above.

As indicated above, in some embodiments, at least some processing of the signal(s) from photodetector 338 is performed at central station 116. Doing so facilitates using additional, more powerful data-processing algorithms to analyze the information contained in the signals.

The information obtained from the signal(s) from photodetector 338 can be used to:
detect biological agents;
estimate the amount of biological agent detected;
determine if the amount of biological agent present is indicative of a biological attack or otherwise poses a risk to the health of the local population, livestock, etc.;
identify the biological agents that are detected.

As to detection, the detection of fluorescence, particularly at certain wavelengths, might be indicative of the presence of a biological agent. The intensity of the signal, as well as the air flow through the interrogation cell and the amount of time that the air has been flowing provides information related to the amount of biological agent present in the environment. In other words, it can be used to develop a particulate count as a function of wavelength. As to identification, the wavelength of fluorescent emissions measured by interrogation cell 106 can be compared to the wavelength of fluorescent emissions of known biological agents. Correspondence between the measured emissions and one of the references is indicative of the presence of that biological agent. For further information about identification of biological agents, see applicants co-pending U.S. patent application Ser. No. ______ (Atty. Dkt. 711-019).

In the illustrative embodiment, the results of signal processing are presented via a graphical user interface. In some embodiments, the results are displayed as an “intensity” or “particle count” as a function of frequency or wavelength of the fluorescent emissions. In some embodiments, an alarm limit is displayed for each “type” (i.e., each different frequency or wavelength) of biological agent. If an alarm limit is exceeded, an alert (e.g., sound, flashing light, etc.) is provided. The manner in which information that is obtained from interrogation cell 106 is presented via a graphical user interface is described in further detail in applicants' co-pending U.S. patent application Ser. No. ______ (Atty. Dkt. 711-016).

Referring once again to FIG. 2, operation 210 recites “repeating operations 202-208 but passing a sample of air through a second sample-collecting region of the collection media.” There are a variety of advantages to using multiple sample-collecting regions, including:
the prevention of excessive particulate build-up, thereby extending run time;
enables off-line, detailed analysis of particulates as a function of collection time or collection location.

FIG. 4 depicts a top view of circular-shaped collection media 330 comprising four sample-collecting regions 344-i, i=1,4. In some embodiments, collection media 330 includes fewer than four sample-collecting regions 344-i, while in some other embodiments, collection media 330 includes more than four sample-collecting regions 344-i. In the illustrative embodiment, sample-collecting regions 344-i are “pie”-shaped segments (i.e., sectors of a circle); in some other embodiments, the sample-collecting regions are not configured in this fashion, whether or not collection media 330 has a circular shape.

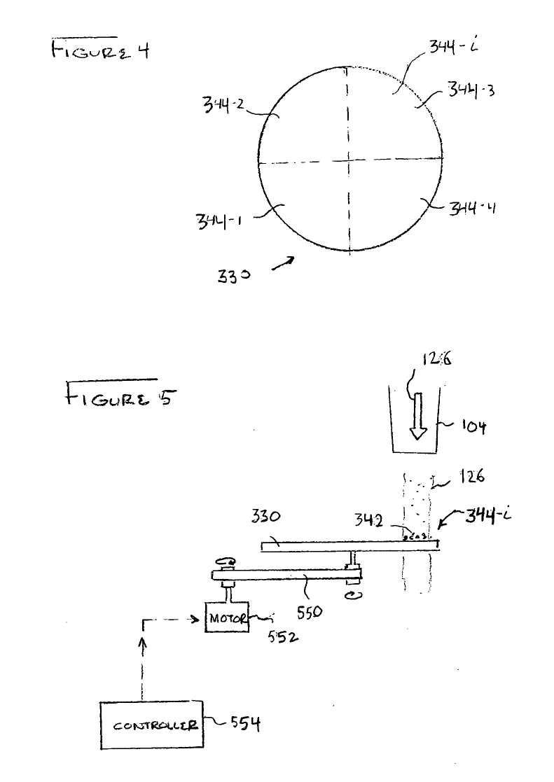

FIG. 5 depicts an embodiment of sensing system 100 suitable for use with collection media 330 having a plurality of sample-collecting regions 344-i. In the embodiment depicted in FIG. 5, collection media 330 is rotatably coupled, via belt 550, to motor 552. Responding to commands from controller 554, motor 552 turns collection media 330 to rotate one of sample-collecting regions 344-i into a sample-receiving position. In the embodiment depicted in FIG. 5, the sample-receiving position aligns with cell inlet line 104. As a consequence, when a particular sample-collecting region 344-i is in the sample-receiving position, it receives flow 126 of air.

At some time, motor 552 is again energized so that the sample-collecting regions 344-i that was in the sample-receiving position is rotated out, and a different sample-collecting regions 344-i is rotated into the sample-receiving position. Each sample-collection region 344-i that has been rotated into the sample-receiving position is “time” stamped (i.e., a particular sample-collection region collected a sample at a certain time) or “location” stamped (i.e., a particular sample-collection region collected a sample when sensing system 100 was at a certain location, etc.). The time stamping can be performed in conjunction with a clock and the location stamping can be performed in conjunction with a global positioning system, VOR, Loran, etc. Stamping is particularly important in embodiments in which collection media 330 is removed from sensing system 100 for post analysis. This facilitates matching up such post analysis with the time(s) or location(s) at which the analyzed sample(s) were obtained.

The time at which motor 552 rotates a different sample-collecting regions 444-i into the sample-receiving position can be based on:
a set time period (e.g., rotate every 30 minutes, etc.);
a command from a sensor that is monitoring the accumulation of particulates within the sample-collecting region (of the region that is receiving the flow of air);
reaching a position/location (in embodiments in which system 100 is being moved in a vehicle);
a command from an operator (e.g., a person that is monitoring the output from system 100);
a random occurrence (e.g., a random time period, etc.).

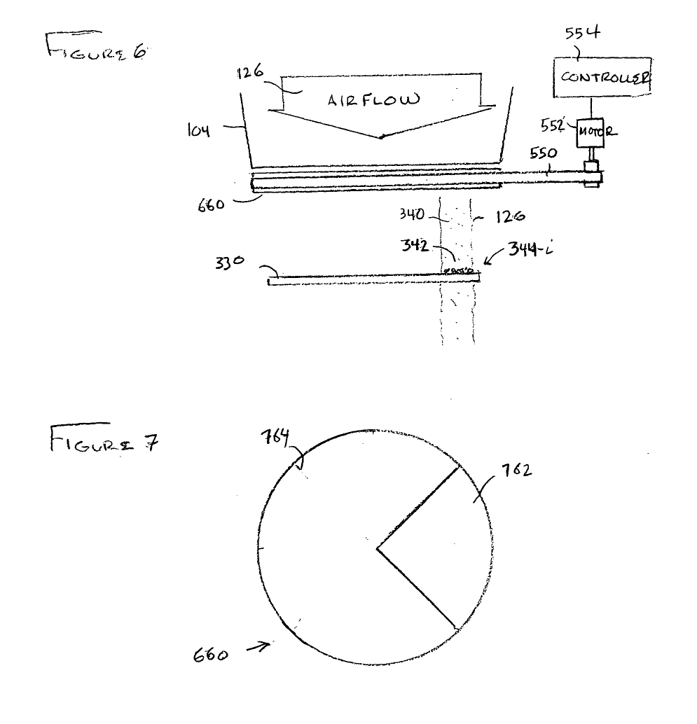

FIG. 6 depicts an alternative embodiment of system 100 suitable for use with collection media 330 having a plurality of sample-collecting regions 344-i. In the embodiment depicted in FIG. 6, collection media 330 is stationary while shutter 660, which is positioned between cell inlet line 104 and collection media 330, is rotated.

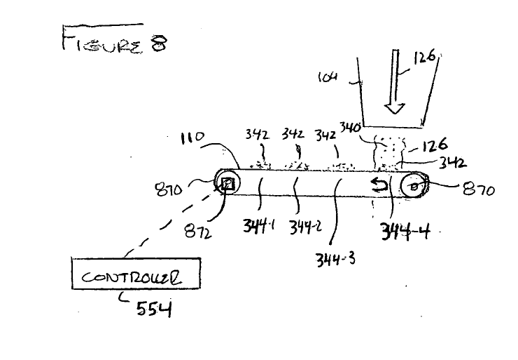

As depicted in FIG. 7, and with continuing reference to FIG. 6, shutter 660 includes opening 762 and solid or closed region 764. All of flow 126 of air from cell inlet line 104 is channeled through opening 762. As a consequence, the particular sample-collecting region 444-i that is positioned “below” opening 762 receives flow 126 of air such that it will be able to extract particulates 340 to form sample 342. In the embodiment depicted in FIG. 6, shutter 660 is rotated by belt 550 in conjunction with motor 552. The motor responds to commands from controller 554, as previously described.

FIG. 8 depicts a further embodiment of sensing system 100 wherein collection media 330 has a plurality of sample-collecting regions 344-i. In the embodiment that is depicted in FIG. 8, collection media 330 is in the form of a belt. Sample-collecting regions 344-1 through 344-4 are spaced locations on collection media 330. Pulleys 870 engage the collection media. At least one of pulleys 870 is driven by motor 872. As described in previous embodiments, motor 872 responds to commands from controller 554.

It is to be understood that the above-described embodiments are merely illustrative of the present invention and that many variations of the above-described embodiments can be devised by those skilled in the art without departing from the scope of the invention. For example, in this Specification, numerous specific details are provided in order provide a thorough description and understanding of the illustrative embodiments of the present invention. Those skilled in the art will recognize, however, that the invention can be practiced without one or more of those details, or with other methods, materials, components, etc. In particular, as appropriate, features that are disclosed in co-pending U.S. patent applications Ser. No. ______ (Attorney Docket Nos. 711-016, 711-018, 711-019, and 711-020) can be used in conjunction with the illustrative embodiment that is depicted and described herein. Those skilled in the art will know how to integrate such features into the illustrative embodiment of the present invention.

In some instances, well-known structures, materials, or operations are not shown or described in detail to avoid obscuring aspects of the illustrative embodiments. It is understood that the various embodiments shown in the Figures are illustrative, and are not necessarily drawn to scale. Reference throughout the specification to “one embodiment” or “an embodiment” or “some embodiments” means that a particular feature, structure, material, or characteristic described in connection with the embodiment(s) is included in at least one embodiment of the present invention, but not necessarily all embodiments. Consequently, the appearances of the phrase “in one embodiment,” “in an embodiment,” or “in some embodiments” in various places throughout the Specification are not necessarily all referring to the same embodiment. Furthermore, the particular features, structures, materials, or characteristics can be combined in any suitable manner in one or more embodiments. It is therefore intended that such variations be included within the scope of the following claims and their equivalents.

We claim:

1. An apparatus comprising:
collection media, wherein said collection media comprises a plurality of stationary-phase sample-collecting regions;
an alignment device, wherein said alignment device aligns sample-collecting regions with a flow of air in a sample-collecting position, wherein, when so aligned, a a sample is collected;
a source of electromagnetic radiation for exposing said sample;
circuitry for intermittently activating said light-emitting-diode; and
a photodetector for detecting fluorescent emissions from said sample resulting from the excitation.

2. The apparatus of claim 1 wherein said collection media comprises teflon®.

3. The apparatus of claim 1 wherein said collection media has a circular shape.

4. The apparatus of claim 3 wherein said sample-collection regions are sectors of said collection media.

5. The apparatus of claim 1 wherein said alignment device comprises a motor for turning said collection media so that said sample-collection regions are sequentially rotated into said sample-collecting position.

6. The apparatus of claim 1 further comprising a device for associating each sample-collecting region that has been aligned with said flow of air in said sample-collecting position with at least one of either a time or a geographic location.

7. The apparatus of claim 1 wherein said alignment device re-directs said flow of air to individually place said sample-collecting regions into said sample-collecting position.

8. The apparatus of claim 7 wherein said alignment device comprises a movable shutter, wherein a position of said movable shutter controls which of said sample-collecting regions receives said flow of air.

9. The apparatus of claim 8 wherein said alignment device comprises a motor for moving said movable shutter.

10. The apparatus of claim 1 comprising control/data-acquisition/data-processing circuitry, wherein said photodetector is electrically coupled to said data-processing circuitry.

11. The apparatus of claim 12 wherein said data-processing circuitry comprises a graphical user interface, wherein said graphical user interface depicts an indicium of an amount of particles in said sample that fluoresce at a first wavelength.

12. The apparatus of claim 13 wherein said graphical user interface provides an indication when said indicium exceeds a maximum acceptable amount.

13. An apparatus comprising:
stationary-phase collection media, wherein said collection media comprises at least a first sample-collecting region and a second sample-collecting region;
a device for enabling said first sample-collecting region to collect a first sample from a first flow of air and for enabling said second sample-collecting region to collect a second sample from a second flow of air, wherein said first sample and said second sample are collected at different times;
a light-emitting diode for exposing said first sample and said second sample to electromagnetic radiation; and
a photodetector for detecting fluorescent emissions from said first sample and said second sample resulting from the exposure to electromagnetic radiation.

14. The apparatus of claim 15 comprising a device for associating each sample-collecting region with at least one of either a time or a geographic location

15. The apparatus of claim 15 comprising a device for directing said first flow of air to said first sample-collecting region and said second flow of air to said second sample-collecting region.

16. The apparatus of claim 17 wherein said device comprises a movable shutter, wherein said movable shutter is disposed between an air intake and said collection media.

17. The apparatus of claim 15 comprising a device for moving said collection media to position, at different times, said first sample-collecting region in a sample-collecting position and said second sample-collecting region in said sample-collecting position.

18. The apparatus of claim 19 wherein said device is a motor.

19. The apparatus of claim 19 comprising a controller, wherein said controller directs said device to move said collection media.

20. A method comprising:
passing a first portion of air through a first sample-collecting region of a collection media;
intermittently exposing said first sample-collecting region with electromagnetic radiation;
detecting fluorescent emissions, caused by the intermittent exposure, from a first group of particulates that were contained in said first portion of air and that are retained in said first sample-collecting region;
passing a second portion of air through a second sample-collecting region of said collection media;
intermittently exposing said second sample-collecting region with electromagnetic radiation; and
detecting fluorescent emissions, caused by the intermittent exposure, from a second group of particulates that were contained in said second portion of air and that are retained in said second sample-collecting region.

21. The method of claim 20 comprising determining an amount of fluorescent emissions having a first wavelength.

22. The method of claim 21 comprising triggering an alert when said determined amount of fluorescent emissions at said first wavelength exceed a first value.

23. The method of claim 21 comprising:
removing said collection media from a movable apparatus;
analyzing said first group of particulates that are retained in said first sample-collecting region using a technique selected from the group consisting of PCR, . . .
